(19) United States
(12) Patent Application Publication
(10) Pub. No.: US 2005/0101485 A1
(43) Pub. Date: May 12, 2005

(54) Methods for the identification of inhibitors of biotin synthase expression or activity in plants

(76) Inventors: Neil Hoffman, Bethesda, MD (US); Jorn Gorlach, Manchester, NJ (US); Douglas Boyes, Chapel Hill, NC (US); Adel Zayed, Durham, NC (US); Jeffrey Woessner, Hillsborough, NC (US); Carol Hamilton, Apex, NC (US); Keith Davis, Durham, NC (US); Robert Ascenzi, Cary, NC (US)

Correspondence Address: Icoria, Inc., 108 T.W. ALEXANDER DRIVE, P O BOX 14528, RTP, NC, 27709-4528, US

(21) Appl. No.: 10/702,364

(22) Filed: Nov. 6, 2003

(51) Int. Cl. C12Q 1/48; C12Q 1/37; A01N 25/00

(52) U.S. Cl. 504/116.1; 435/23

(57) ABSTRACT

The present inventors have discovered that Biotin synthase (BS) is essential for plant growth. Specifically, inhibition of BS gene expression in plant seedlings results in severe chlorosis and reduced growth. Thus, BS can be used as a target for the identification of herbicides. Accordingly, the present invention provides methods for the identification of compounds that inhibit BS expression or activity, and as such, the methods of the invention are useful for the identification of herbicides.

This application is the national phase under 35 U.S.C. § 365 of PCT International Application No. PCT/US02/14473, that has an International filing date of May 7, 2002, which designated the United States of America and which claims the benefit of U.S. Provisional Application Ser. No. 60/289,312, filed May 7, 2001.

### FIELD OF THE INVENTION

The invention relates generally to plant molecular biology. In particular, the invention relates to methods for the identification of herbicides.

### BACKGROUND OF THE INVENTION

Biotin synthase (“BS”) from plants has been fairly well characterized. Biotin synthase (bioB, BIO2, BS) (EC 2.8.1.6) is involved in the conversion of dethiobiotin to biotin in bacteria, yeast, and higher plants. Bui et al., 440 FEBS LETT. 226-30 (1998) (PMID: 9862460); Baldet et al., 217 EUR. J. BIOCHEM 479-85 (1993) (PMID: 8223585); and Baldet et al., 319 CR ACAD. SCI. III 99-106 (1996) (PMID: 8680961). This enzymatic reaction involves the unusual addition of sulfur to form a thiophene ring. Ollagnier-de Choudens et al., 453 FEBS LETT. 25-28 (1999) (PMID: 10403368). Isolation of a complete biotin synthase Arabidopsis cDNA was first reported in 1996. Baldet et al., 319 CR ACAD. SCI. III 99-106 (1996) (PMID: 8680961). The predicted amino acid sequence of the plant protein contains the consensus region GXCXEDCXYCXQ involved in the [2Fe-2S] cluster binding. Id. The threonine-173 residue, which is highly conserved in biotin synthases, was further shown to be important for catalytic competence of the enzyme through site-specific mutagenesis. Weaver et al., 110 PLANT PHYSIOL. 1021-28 (1996) (PMID: 8819873). The primary sequence of the Arabidopsis biotin synthase is most similar to biotin synthases from E. coli, Serratia marcescens, and Saccharomyces cerevisiae (about 50% sequence identity) and more distantly related to the Bacillus sphaericus enzyme (33% sequence identity).

BIO2 (BS) is a single-copy nuclear gene in Arabidopsis that is expressed at high levels in the tissues of immature plants. Expression of BIO2 was higher in the light relative to dark and was induced 5-fold during biotin-limited conditions. These results demonstrate that expression of at least one gene in this pathway is regulated in response to developmental, environmental, and bio-chemical stimuli. Patton et al., 112 PLANT PHYSIOL. 371-78 (1996) (PMID: 8819333). The purified A. thaliana bioB gene product is a homodimer (100 kDa) with a reddish color and has an absorbance spectrum characteristic of protein with [2Fe-2S] clusters. Its intracellular compartmentation in pea leaves discloses a unique polypeptide of 39 kDa within the matrix of mitochondria. Baldet et al., 419 FEBS LETT. 206-10 (1997) (PMID: 9428635).

Patton et al. have identified arrested embryos from a bio2 (BS) mutant defective in the final step of biotin synthesis. Patton et al., 116 PLANT PHYSIOL. 935-46 (1996) (PMID: 9501126). However, the literature in no way describes the lethal effects of over-expression, antisense expression, or knock-out of the BS gene in plants. Accordingly, the prior art has not suggested that BS is essential for plant growth and development. It would be highly desirable to use the BS enzyme for evaluating and determining its effect on plant regulation and growth, and therefor, in identifying and evaluating compounds as having herbicidal activity.

### SUMMARY OF THE INVENTION

The present inventors have discovered that antisense expression of a biotin synthase complementary DNA (BS cDNA) in Arabidopsis causes developmental abnormalities, and short and extremely stunted plant seedlings. Thus, the present inventors have discovered that BS is essential for normal seed development and growth, and can be used as a target for the identification of herbicides. Accordingly, the present invention provides methods for the identification of compounds that inhibit BS expression or activity, comprising: contacting a candidate compound with a BS and detecting the presence or absence of binding between the compound and the BS, or detecting a decrease in BS expression or activity. The methods of the invention are useful for the identification of herbicides.

### BRIEF DESCRIPTION OF THE DRAWINGS

FIG. 1 shows the biotin synthase reaction.

### DETAILED DESCRIPTION OF THE INVENTION

Definitions

The term “binding” refers to a noncovalent interaction that holds two molecules together. For example, two such molecules could be an enzyme and an inhibitor of that enzyme. Noncovalent interactions include hydrogen bonding, ionic interactions among charged groups, van der Waals interactions and hydrophobic interactions among nonpolar groups. One or more of these interactions can mediate the binding of two molecules to each other.

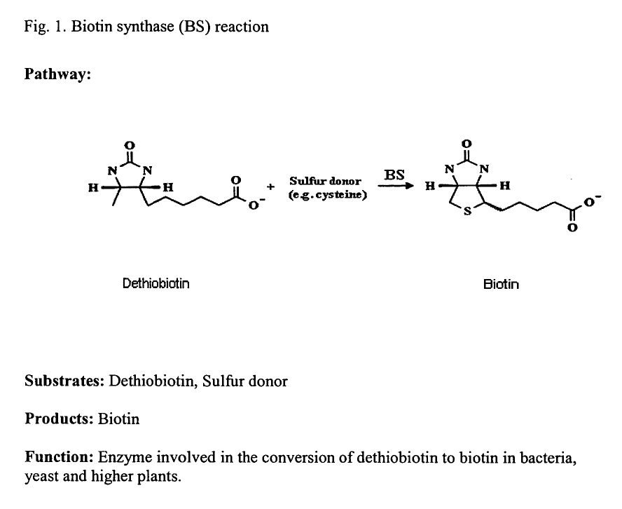

As used herein, the term “Biotin synthase (EC 2.8.1.6)” is synonymous with “BS” and refers to an enzyme that catalyses the conversion of dethiobiotin and a sulfur donor to biotin, as shown in FIG. 1. The cDNA (SEQ ID No. 1) encoding the BS polypeptide or protein (SEQ ID No. 2) is found herein as well as in the TIGR database at locus T1024.10.

The term “herbicide,” as used herein, refers to a compound that may be used to kill or suppress the growth of at least one plant, plant cell, plant tissue or seed.

The term “inhibitor,” as used herein, refers to a chemical substance that inactivates the enzymatic activity of AS. The inhibitor may function by interacting directly with the enzyme, a cofactor of the enzyme, the substrate of the enzyme, or any combination thereof.

A polynucleotide may be “introduced” into a plant cell by any means, including transfection, transformation or transduction, electroporation, particle bombardment, agroinfection and the like. The introduced polynucleotide may be maintained in the cell stably if it is incorporated into a non-chromosomal autonomous replicon or integrated into the plant chromosome. Alternatively, the introduced polynucleotide may be present on an extra-chromosomal non-replicating vector and be transiently expressed or transiently active.

The “percent (%) sequence identity” between two polynucleotide or two polypeptide sequences is determined according to the either the BLAST program (Basic Local Alignment Search Tool: Altschul & Gish, 266 METH. ENZYMOL. 460-480 (1996); Altschul, 215 J. MOL. BIOL. 403-10 (1990)) in the Wisconsin Genetics Software Package (Devererreux et al., 12 NUCL. ACID RES. 387 (1984)), Genetics Computer Group, Madison, Wis. (NCBI, Version 2.0.11, default settings) or using Smith Waterman Alignment (Smith & Waterman, 2 ADV. APPL. MATH 482 (1981)) as incorporated into GENEMATCHER PLUS (Paracel, Inc., Internet-accessible interface using the default settings and the version current at the time of filing). It is understood that for the purposes of determining sequence identity when comparing a DNA sequence to an RNA sequence, a thymine nucleotide is equivalent to a uracil nucleotide.

“Plant” refers to whole plants, plant organs and tissues (e.g., stems, roots, ovules, stamens, leaves, embryos, meristematic regions, callus tissue, gametophytes, sporophytes, pollen, microspores and the like) seeds, plant cells, and the progeny thereof.

By “polypeptide” is meant a chain of at least four amino acids joined by peptide bonds. The chain may be linear, branched, circular, or combinations thereof. The polypeptides may contain amino acid analogs and other modifications, including, but not limited to glycosylated or phosphorylated residues.

The term “specific binding” refers to an interaction between BS and a molecule or compound, wherein the interaction is dependent upon the primary amino acid sequence or the conformation of BS.

Embodiments of the Invention

The present inventors have discovered that inhibition of BS gene expression strongly inhibits the growth and development of plant seedlings. Thus, the inventors are the first to demonstrate that BS is a target for herbicides.

Accordingly, the invention provides methods for identifying compounds that inhibit BS gene expression or activity. Such methods include ligand-binding assays, assays for enzyme activity and assays for BS gene expression. Any compound that is a ligand for BS, other than its substrates, dethiobiotin and a sulfur donor, may have herbicidal activity. For the purposes of the invention, “ligand” refers to a molecule that will bind to a site on a polypeptide. The compounds identified by the methods of the invention are useful as herbicides.

The sulfur donor may be, but is not limited to, cysteine, sulfur, glutathione, N-acetyl cysteine, methionine, cystine, 4,4′-dithio-bis-morpholine, dithiodicaprolactam, alkylphenol disulfide, or amylphenol disulfide. Preferably the sulfur donor is cysteine.

Thus, in one embodiment, the invention provides a method for identifying a compound as a candidate for a herbicide, comprising:

a) contacting a BS with the compound; and

b) detecting the presence and/or absence of binding between the compound and the BS, wherein binding indicates that the compound is a candidate for a herbicide.

By “BS” is meant any enzyme that catalyzes the interconversion of dethiobiotin and a sulfur donor with biotin. The BS may have the amino acid sequence of a naturally occurring BS found in a plant, animal or microorganism, or may have an amino acid sequence derived from a naturally occurring sequence. Preferably the BS is a plant BS.

By “plant BS” is meant an enzyme that can be found in at least one plant, and which catalyzes the interconversion of dethiobiotin and a sulfur donor with biotin. The BS may be from any plant, including both monocots and dicots.

In one embodiment, the BS is an Arabidopsis BS. Arabidopsis species include, but are not limited to, Arabidopsis arenosa, Arabidopsis bursifolia, Arabidopsis cebennensis, Arabidopsis croatica, Arabidopsis griffithiana, Arabidopsis halleri, Arabidopsis himalaica, Arabidopsis korshinskyi, Arabidopsis lyrata, Arabidopsis neglecta, Arabidopsis pumila, Arabidopsis suecica, Arabidopsis thaliana and Arabidopsis wallichii. Preferably, the Arabidopsis BS is from Arabidopsis thaliana.

In various embodiments, the BS can be from barnyard grass (Echinochloa crus-galli), crabgrass (Digitaria sanguinalis), green foxtail (Setana viridis), perennial ryegrass (Lolium perenne), hairy beggarticks (Bidens pilosa), nightshade (Solanum nigrum), smartweed (Polygonuni lapathifolium), velvetleaf (Abutilon theophrasti), common lambsquarters (Chenopodium album L.), Brachiara plantaginea, Cassia occidentalis, Ipomoea aristolochiaefolia, Ipomoea purpurea, Euphorbia heterophylla, Setaria spp, Amaranthus retroflexus, Sida spinosa, Xanthium strumarium and the like.

Fragments of a BS polypeptide may be used in the methods of the invention. The fragments comprise at least 10 consecutive amino acids of a BS. Preferably, the fragment comprises at least 15, 20, 25, 30, 35, 40, 50, 60, 70, 80, 90 or at least 100 consecutive amino acids residues of a BS. In one embodiment, the fragment is from an Arabidopsis BS. Preferably, the fragment contains an amino acid sequence conserved among plant biotin synthases. Such conserved fragments have been previously reported. (Grima-Pettenuti et al., 21 PLANT MOL. BIOL. 1085-1095 (1993)). Those skilled in the art could identify additional conserved fragments using sequence comparison software.

Polypeptides having at least 80% sequence identity with a plant BS are also useful in the methods of the invention. Preferably, the sequence identity is at least 85%, more preferably the identity is at least 90%, most preferably the sequence identity is at least 95% or 99%.

In addition, it is preferred that the polypeptide has at least 50% of the activity of a plant BS. More preferably, the polypeptide has at least 60%, at least 70%, at least 80% or at least 90% of the activity of a plant BS. Most preferably, the polypeptide has at least 50%, at least 60%, at least 70%, at least 80% or at least 90% of the activity of the A. thaliana BS protein.

Thus, in another embodiment, the invention provides a method for identifying a compound as a candidate for a herbicide, comprising:

a) contacting the compound with at least one polypeptide selected from the group consisting of: a plant BS, a polypeptide comprising at least ten consecutive amino acids of a plant BS, a polypeptide having at least 85% sequence identity with a plant BS, and a polypeptide having at least 80% sequence identity with a plant BS and at least 50% of the activity thereof; and

b) detecting the presence and/or absence of binding between the compound and the polypeptide, wherein binding indicates that the compound is a candidate for a herbicide.

Any technique for detecting the binding of a ligand to its target may be used in the methods of the invention. For example, the ligand and target are combined in a buffer. Many methods for detecting the binding of a ligand to its target are known in the art, and include, but are not limited to the detection of an immobilized ligand-target complex or the detection of a change in the properties of a target when it is bound to a ligand. For example, in one embodiment, an array of immobilized candidate ligands is provided. The immobilized ligands are contacted with a BS protein or a fragment or variant thereof, the unbound protein is removed and the bound BS is detected. In a preferred embodiment, bound BS is detected using a labeled binding partner, such as a labeled antibody. In a variation of this assay, BS is labeled prior to contacting the immobilized candidate ligands. Preferred labels include fluorescent or radioactive moieties. Preferred detection methods include fluorescence correlation spectroscopy (“FCS”) and FCS-related confocal nanofluorimetric methods.

Once a compound is identified as a candidate for a herbicide, it can be tested for the ability to inhibit BS enzyme activity. The compounds can be tested using either in vitro or cell based enzyme assays. Alternatively, a compound can be tested by applying it directly to a plant or plant cell, or expressing it therein, and monitoring the plant or plant cell for changes or decreases in growth, development, viability or alterations in gene expression.

Thus, in one embodiment, the invention provides a method for determining whether a compound identified as a herbicide candidate by an above method has herbicidal activity, comprising: contacting a plant or plant cells with the herbicide candidate and detecting the presence or absence of a decrease in the growth or viability of the plant or plant cells.

By decrease in growth, is meant that the herbicide candidate causes at least a 10% decrease in the growth of the plant or plant cells, as compared to the growth of the plants or plant cells in the absence of the herbicide candidate. By a decrease in viability is meant that at least 20% of the plants cells, or portion of the plant contacted with the herbicide candidate are nonviable. Preferably, the growth or viability will be at decreased by at least 40%. More preferably, the growth or viability will be decreased by at least 50%, 75% or at least 90% or more. Methods for measuring plant growth and cell viability are known to those skilled in the art. It is possible that a candidate compound may have herbicidal activity only for certain plants or certain plant species.

The ability of a compound to inhibit BS activity can be detected using in vitro enzymatic assays in which the disappearance of a substrate or the appearance of a product is directly or indirectly detected. BS catalyzes the irreversible or reversible reaction of dethiobiotin and a sulfur donor to biotin. Methods for detection of dethiobiotin, a sulfur donor, and/or biotin, include spectrophotometry, mass spectroscopy, thin layer chromatography (“TLC”) and reverse phase HPLC.

Thus, the invention provides a method for identifying a compound as a candidate for a herbicide, comprising:
a) contacting a dethiobiotin and a sulfur donor with BS;
b) contacting the dethiobiotin and a sulfur donor with BS and the candidate compound; and
c) determining the concentration of biotin after the contacting of steps (a) and (b).

If a candidate compound inhibits BS activity, a higher concentration of the substrates (dethiobiotin and a sulfur donor) and a lower level of the product (biotin) will be detected in the presence of the candidate compound (step b) than in the absence of the compound (step a).

Preferably the BS is a plant BS. Enzymatically active fragments of a plant BS are also useful in the methods of the invention. For example, a polypeptide comprising at least 100 consecutive amino acid residues of a plant BS may be used in the methods of the invention. In addition, a polypeptide having at least 80%, 85%, 90%, 95%, 98% or at least 99% sequence identity with a plant BS may be used in the methods of the invention. Preferably, the polypeptide has at least 80% sequence identity with a plant BS and at least 50%, 75%, 90% or at least 95% of the activity thereof.

Thus, the invention provides a method for identifying a compound as a candidate for a herbicide, comprising:
a) contacting dethiobiotin and a sulfur donor with a polypeptide selected from the group consisting of: a polypeptide having at least 85% sequence identity with a plant BS, a polypeptide having at least 80% sequence identity with a plant BS and at least 50% of the activity thereof, and a polypeptide comprising at least 100 consecutive amino acids of a plant BS;
b) contacting the dethiobiotin and a sulfur donor with the polypeptide and the compound; and
c) determining the concentration of biotin after the contacting of steps (a) and (b).

Again, if a candidate compound inhibits BS activity, a higher concentration of the substrate (dethiobiotin and a sulfur donor) and a lower level of the product (biotin) will be detected in the presence of the candidate compound (step b) than in the absence of the compound (step a).

For the in vitro enzymatic assays, BS protein and derivatives thereof may be purified from a plant or may be recombinantly produced in and purified from a plant, bacteria, or eukaryotic cell culture. Preferably these proteins are produced using a baculovirus or E. coli expression system. Methods for the purification of biotin synthase have been described. Baldet et al., 319 CR ACAD. SCI. III 99-106 (1996) (PMID: 8680961). Other methods for the purification of BS proteins and polypeptides are known to those skilled in the art.

As an alternative to in vitro assays, the invention also provides plant and plant cell based assays. In one embodiment, the invention provides a method for identifying a compound as a candidate for a herbicide, comprising:

a) measuring the expression of BS in a plant or plant cell in the absence of the compound;

b) contacting a plant or plant cell with the compound and measuring the expression of BS in the plant or plant cell; and

c) comparing the expression of BS in steps (a) and (b).

A reduction in BS expression indicates that the compound is a herbicide candidate. In one embodiment, the plant or plant cell is an Arabidopsis thaliana plant or plant cell.

Expression of BS can be measured by detecting BS primary transcript or mRNA, BS polypeptide or BS enzymatic activity. Methods for detecting the expression of RNA and proteins are known to those skilled in the art. See e.g., Current Protocols in Molecular Biology, (Ausubel et al., eds., Greene Publishing and Wiley-Interscience) (1995). The method of detection is not critical to the invention. Methods for detecting BS RNA include, but are not limited to amplification assays such as quantitative PCR, and/or hybridization assays such as Northern analysis, dot blots, slot blots, in-situ hybridization, transcriptional fusions using a BS promoter fused to a reporter gene, bDNA assays and microarray assays.

Methods for detecting protein expression include, but are not limited to, immunodetection methods such as Western blots, His Tag and ELISA assays, polyacrylamide gel electrophoresis, mass spectroscopy and enzymatic assays. Also, any reporter gene system may be used to detect BS protein expression. For detection using gene reporter systems, a polynucleotide encoding a reporter protein is fused in frame with BS, so as to produce a chimeric polypeptide. Methods for using reporter systems are known to those skilled in the art. Examples of reporter genes include, but are not limited to: chloramphenicol acetyltransferase (Gorman et al., 2 MOL. CELL BIOL. 1104 (1982); Prost et al., 45 GENE 107-111 (1986)); β-galactosidase (Nolan et al., 85 PROC. NAT. ACAD. SCI. USA 2603-2607 (1988)); alkaline phosphatase (Berger et al., 66 GENE 10 (1988)); luciferase (De Wet et al., 7 MOL. CELL BIOL. 725-737 (1987)); β-glucuronidase (“GUS”); fluorescent proteins; chromogenic proteins, and the like. Methods for detecting BS activity are described above.

Chemicals, compounds, or compositions identified by the above methods as modulators of BS expression or activity can then be used to control plant growth. For example, compounds that inhibit plant growth can be applied to a plant or expressed in a plant, in order to prevent plant growth. Thus, the invention provides a method for inhibiting plant growth, comprising contacting a plant with a compound identified by the methods of the invention as having herbicidal activity.

Herbicides and herbicide candidates identified by the methods of the invention can be used to control the growth of undesired plants, including both monocots and dicots. Examples of undesired plants include, but are not limited to barnyard grass (Echinochloa crus-galli), crabgrass (Digitaria sanguinalis), green foxtail (Setana viridis), perennial ryegrass (Lolium perenne), hairy beggarticks (Bidens pilosa), nightshade (Solanum nigrum), smartweed (Polygonum lapathifolium), velvetleaf (Abutilon theophrasti), common lambsquarters (Chenopodium album L.), Brachiara plantaginea, Cassia occidentalis, Ipomoea aristolochiaefolia, Ipomoea purpurea, Euphorbia heterophylla, Setaria spp, Amaranthus retroflexus, Sida spinosa, Xanthium strumarium and the like.

### Experimental

Plant Growth Conditions

Unless, otherwise indicated, all plants are grown Scotts Metro-Mix™ soil (the Scotts Company) or a similar soil mixture in an environmental growth room at 22° C., 65% humidity, 65% humidity and a light intensity of ˜100 μ-E m⁻² s⁻¹ supplied over 16 hour day period.

Seed Sterilization

All seeds are surface sterilized before sowing onto phytagel plates using the following protocol.

1. Place approximately 20-30 seeds into a labeled 1.5 ml conical screw cap tube. Perform all remaining steps in a sterile hood using sterile technique.

2. Fill each tube with 1 ml 70% ethanol and place on rotisserie for 5 minutes.

3. Carefully remove ethanol from each tube using a sterile plastic dropper; avoid removing any seeds.

4. Fill each tube with 1 ml of 30% bleach and 0.5% SDS solution and place on rotisserie for 10 minutes.

5. Carefully remove bleach/SDS solution.

6. Fill each tube with 1 ml sterile dI H₂O; seeds should be stirred up by pipetting of water into tube. Carefully remove water. Repeat 3 to 5 times to ensure removal of bleach/SDS solution.

7. Fill each tube with enough sterile dI H₂O for seed plating (˜200-400 μl). Cap tube until ready to begin seed plating.

Plate Growth Assays

Surface sterilized seeds are sown onto plate containing 40 ml half strength sterile MS (Murashige and Skoog, no sucrose) medium and 1% Phytagel using the following protocol:

1. Using pipette and 200 μl tip, carefully fill tip with seed solution. Place 10 seeds across the top of the plate, about ¼ in down from the top edge of the plate.

2. Place plate lid ¾ of the way over the plate and allow to dry for 10 minutes.

3. Using sterile micropore tape, seal the edge of the plate where the top and bottom meet.

4. Place plates stored in a vertical rack in the dark at 4° C. for three days.

5. Three days after sowing, the plates transferred into a growth chamber with a day and night temperature of 22° C. and 20° C., respectively, 65% humidity and a light intensity of ˜100 μ-E m⁻² s⁻¹ supplied over 16 hour day period.

6. Beginning on day 3, daily measurements are carried out to track the seedlings development until day 14. Seedlings are harvested on day 14 (or when root length reaches 6 cm) for root and rosette analysis.

### EXAMPLE 1

### Construction of a Transgenic Plant Expressing the Driver

The “Driver” is an artificial transcription factor comprising a chimera of the DNA-binding domain of the yeast GAL4 protein (amino acid residues 147) fused to two tandem activation domains of herpes simplex virus protein VP16 (amino acid residues 413-490). Schwechheimer et al., 36 PLANT MOL. BIOL. 195-204 (1998). This chimeric driver is a transcriptional activator specific for promoters having GAL4 binding sites. Expression of the driver is controlled by two tandem copies of the constitutive CaMV ³⁵S promoter.

The driver expression cassette is introduced into Arabidopsis thaliana by agroinfection. Transgenic plants that stably expressed the driver transcription factor are obtained.

### EXAMPLE 2

### Construction of Antisense Expression Cassettes in a Binary Vector

A fragment, fragment or variant of an Arabidopsis thaliana cDNA corresponding to SEQ ID NO:1 is ligated into the PacI/AscI sites of an E.coli/Agrobacterium binary vector in the antisense orientation. This places transcription of the antisense RNA under the control of an artificial promoter that is active only in the presence of the driver transcription factor described above. The artificial promoter contains four contiguous binding sites for the GAL4 transcriptional activator upstream of a minimal promoter comprising a TATA box.

The ligated DNA is transformed into E. coli. Kanamycin resistant clones are selected and purified. DNA is isolated from each clone and characterized by PCR and sequence analysis. The DNA is inserted in a vector that expresses the A. thaliana antisense RNA, which is complementary to a portion of the DNA of SEQ ID NO:1. This antisense RNA is complementary to the cDNA sequence found in the TIGR database at locus T1024.10. The coding sequence for this locus is shown as SEQ ID NO:1. The protein encoded by these mRNAs is shown as SEQ ID NO:2.

The antisense expression cassette and a constitutive chemical resistance expression cassette are located between right and left T-DNA borders. Thus, the antisense expression cassettes can be transferred into a recipient plant cell by agroinfection.

### EXAMPLE 3

### Transformation of Agrobacterium with the Antisense Expression Cassette

The vector is transformed into Agrobacteriun tumefaciens by electroporation. Transformed Agrobacterium colonies are isolated using chemical selection. DNA is prepared from purified resistant colonies and the inserts are amplified by PCR and sequenced to confirm sequence and orientation.

### EXAMPLE 4

### Construction of an Arabidopsis Antisense Target Plants

The antisense expression cassette is introduced into Arabidopsis thaliana wild-type plants by the following method. Five days prior to agroinfection, the primary inflorescence of Arabidopsis thaliana plants grown in 2.5 inch pots are clipped in order enhance the emergence of secondary bolts.

At two days prior to agroinfection, 5 ml LB broth (10 g/L Peptone, 5 g/L Yeast extract, 5 g/L NaCl, pH 7.0 plus 25 mg/L kanamycin added prior to use) is inoculated with a clonal glycerol stock of Agrobacterium carrying the desired DNA. The cultures are incubated overnight at 28° C. at 250 rpm until the cells reached stationary phase. The following morning, 200 ml LB in a 500 ml flask is inoculated with 500 μl of the overnight culture and the cells are grown to stationary phase by overnight incubation at 28° C. at 250 rpm. The cells are pelleted by centrifugation at 8000 rpm for 5 minutes. The supernatant is removed and excess media is removed by setting the centrifuge bottles upside down on a paper towel for several minutes. The cells are then resuspended in 500 ml infiltration medium (autoclaved 5% sucrose) and 250 μl/L SILWET L-77 (84% polyalkyleneoxide modified heptamethyltrisiloxane and 16% allyloxypolyethyleneglycol methyl ether), and transferred to a one liter beaker.

The previously clipped Arabidopsis plants are dipped into the Agrobacterium suspension so that all above ground parts are immersed and agitated gently for 10 seconds. The dipped plants are then cover with a tall clear plastic dome in order to maintain the humidity, and returned to the growth room. The following day, the dome is removed and the plants are grown under normal light conditions until mature seeds are produced. Mature seeds are collected and stored desiccated at 4° C.

Transgenic Arabidopsis T1 seedlings are selected. Approximately 70 mg seeds from an agrotransformed plant are mixed approximately 4:1 with sand and placed in a 2 ml screw cap cryo vial.

One vial of seeds is then sown in a cell of an 8 cell flat. The flat is covered with a dome, stored at 4° C. for 3 days, and then transferred to a growth room. The domes are removed when the seedlings first emerged. After the emergence of the first primary leaves, the flat is sprayed uniformly with a herbicide corresponding to the chemical resistance marker plus 0.005% SILWET (50 μl/L) until the leaves are completely wetted. The spraying is repeated for the following two days.

Ten days after the first spraying resistant plants are transplanted to 2.5 inch round pots containing moistened sterile potting soil. The transplants are then sprayed with herbicide and returned to the growth room. These herbicide resistant plants represent stably transformed T1 plants.

### EXAMPLE 5

### Effect of Antisense Expression in Arabidopsis Seedlings

The T1 antisense target plants from the transformed plant lines obtained in Example 4 were crossed with the Arabidopsis transgenic driver line described above. The resulting F1 seeds were then subjected to a PGI plate assay to observe seedling growth over a 2-week period. Seedlings were inspected for growth and development. The transgenic plant line containing the antisense construct exhibited significant developmental abnormalities during early development. A clear 1:1 segregation ratio was observed in 2 antisense lines demonstrating that the antisense expression of the gene results in significantly impaired growth and represents an essential gene for normal plant growth and development. Two transgenic lines containing the antisense construct for argininosuccinate synthase exhibited significant seedling abnormalities. Seedlings had chlorotic cotyledons, and red patches on the abaxial sides of the cotyledons.

### EXAMPLE 6

### Cloning and Expression Strategies, Extraction and Purfication of the AS Protein

The following protocol may be employed to obtain the purified AS protein.

Cloning and Expression Strategies:
BS gene can be cloned into E. coli (pET vectors-Novagen), Baculovirus (Pharmingen) and Yeast (Invitrogen) expression vectors containing His/fusion protein tags. Evaluate the expression of recombinant protein by SDS-PAGE and Western blot analysis.

Extraction:
Extract recombinant protein from 250 ml cell pellet in 3 ml of extraction buffer, by sonicating 6 times, with 6 sec pulses at 4° C. Centrifuge extract at 15000×g for 10 min and collect supernatant. Assess biological activity of the recombinant protein by activity assay.

Purification:
Purify recombinant protein by Ni-NTA affinity chromatography (Quiagen).
Purification protocol: perform all steps at 4° C.:
   Use 3 ml Ni-beads
   Equilibrate column with the buffer
   Load protein extract
   Wash with the equilibration buffer
   Elute bound protein with 0.5 M imidazole

### EXAMPLE 7

### Assays for Testing Inhibitors or Candidates for Inhibition of BS Activity

The enzymatic activity of BS may be determined in the presence and absence of candidate inhibitors in a suitable reaction mixture, such as described by the following known assay protocols and others known in the art:

A. Radiochemical Assay:
This assay is based on the conversion of [¹⁴C] dethiobiotin and a sulfur donor to [¹⁴C] biotin. Birch et al., 275 J. BIO. CHEM. 32277-80 (2000).

B. Liquid Chromatography-Mass Spectrometry Assay:
This assay is based on the separation and quantitation of dethiobiotin and biotin. Azoulay et al., 26 J. CHROMATOGRAPHY 272-76 (1984).

While the foregoing describes certain embodiments of the invention, it will be understood by those skilled in the art that variations and modifications may be made and still fall within the scope of the invention.

1. A method for identifying a compound as a candidate for a herbicide, comprising:
a) contacting a BS with a compound; and
b) detecting the presence and/or absence of binding between the compound and the BS, wherein binding indicates that the compound is a candidate for a herbicide.

2. The method of claim 1, wherein the BS is a plant BS.

3. The method of claim 2, wherein the BS is an Arabidopsis BS.

4. The method of claim 3, wherein the BS is SEQ ID. NO. 2.

5. A method for determining whether a compound identified as a herbicide candidate by the method of claim 1 has herbicidal activity, comprising: contacting a plant or plant cells with the herbicide candidate and detecting the presence or absence of a decrease in growth or viability of the plant or plant cells.

6. A method for identifying a compound as a candidate for a herbicide, comprising:
a) contacting a compound with at least one polypeptide selected from the group consisting of: an amino acid sequence comprising at least ten consecutive amino acids of a plant AS, an amino acid sequence having at least 85% sequence identity with a plant BS, and an amino acid sequence having at least 80% sequence identity with a plant BS and at least 50% of the activity thereof; and
b) detecting the presence and/or absence of binding between the compound and the polypeptide, wherein binding indicates that the compound is a candidate for a herbicide.

7. A method for determining whether a compound identified as a herbicide candidate by the method of claim 6 has herbicidal activity, comprising: contacting a plant or plant cells with the herbicide candidate and detecting the presence or absence of a decrease in growth or viability of the plant or plant cells.

8. A method for identifying a compound as a candidate for a herbicide, comprising:
a) contacting a dethiobiotin and a sulfur donor with BS;
b) contacting the dethiobiotin and a sulfur donor with BS and the candidate compound; and
c) determining the concentration of at least one of dethiobiotin, a sulfur donor, and/or biotin after the contacting of steps (a) and (b).

9. The method of claim 8, wherein the BS is a plant BS.

10. The method of claim 9, wherein the BS is an Arabidopsis BS.

11. The method of claim 10, wherein the BS is SEQ ID. NO. 2.

12. A method for identifying a compound as a candidate for a herbicide, comprising:
a) contacting dethiobiotin and a sulfur donor with a polypeptide selected from the group consisting of: a polypeptide having at least 85% sequence identity with a plant BS, a polypeptide having at least 80% sequence identity with a plant BS and at least 50% of the activity thereof, and a polypeptide comprising at least 100 consecutive amino acids of a plant BS;
b) contacting the dethiobiotin and a sulfur donor with the polypeptide and the compound; and
c) determining the concentration of at least one of dethiobiotin, a sulfur donor, and/or biotin after the contacting of steps (a) and (b).

13. A method for identifying a compound as a candidate for a herbicide, comprising:
a) measuring the expression of a BS in a plant or plant cell in the absence of a compound;
b) contacting a plant or plant cell with the compound and measuring the expression of the BS in the plant or plant cell;
c) comparing the expression of BS in steps (a) and (b).

14. The method of claim 13 wherein the plant or plant cell is an Arabidopsis plant or plant cell.

15. The method of claim 14, wherein the BS is SEQ ID NO 2.

16. The method of claim 13, wherein the expression of BS is measured by detecting BS mRNA.

17. The method of claim 13, wherein the expression of BS is measured by detecting BS polypeptide.


SEQUENCE LISTING (2 sequences)

SEQ ID NO: 1 - DNA, 1137 bp, Arabidopsis thaliana
  misc_feature: cDNA TIGR database locus T1024.10
atgatgcttgttcgatctgtatttcgatctcagttgcgaccctctgtctcgggtggtctgcaatctgcttcttgctattcttcattatctgctgcttcagctgaagctgagaggactatcagagaaggtcccagaaacgattggagtagagatgaaatcaagtctgtttatgattctcctcttcttgacctcctcttccatggagctcaggttcatagacatgttcataacttcagggaggtacaacaatgtaccctcctctccataaagactggtggctgtagtgaagactgttcatattgtcctcagtcttcgagatatagcactggagttaaggcacaaagactcatgtctaaggacgctgtcattgatgctgctaagaaggcaaaagaagctgggagcacacgtttttgcatgggtgctgcttggcgagatacaattggacggaaaaccaacttcagccagattcttgaatacatcaaagaaataagaggcatggggatggaagtttgctgcaccttaggcatgattgagaaacaacaagcactagagctaaagaaggctggcctcactgcttataaccacaatcttgatacttcaagagagtactacccaaacgtcatcactactagaagttatgacgatcgccttgaaactcttagccatgttcgtgatgctggaatcaacgtttgttcaggaggaatcatagggcttggtgaggcagaggaagacagaataggtttattacacacgctggcaacacttccttctcaccctgagagtgttcccattaatgctctacttgcagtgaaaggcactcctcttgaagaccagaagccagttgagatatgggagatgatcaggatgattggaaccgcacgtattgtaatgccaaaagcgatggtgagactgtctgctggtagagtccggttctcaatgtccgaacaagctctctgtttccttgctggtgcaaactctatcttcaccggagagaagcttttaaccacaccaaacaatgattttgacgctgaccagctcatgttcaagacattaggcctcattcctaaaccgccaagtttctctgaagatgattctgaatcagaaaactgcgagaaagttgcttccgcttctcactaa

SEQ ID NO: 2 - protein, 378 aa, Arabidopsis thaliana
MMLVRSVFRSQLRPSVSGGLQSASCYSSLSAASAEAERTIREGPRNDWSRDEIKSVYDSPLLDLLFHGAQVHRHVHNFREVQQCTLLSIKTGGCSEDCSYCPQSSRYSTGVKAQRLMSKDAVIDAAKKAKEAGSTRFCMGAAWRDTIGRKTNFSQILEYIKEIRGMGMEVCCTLGMIEKQQALELKKAGLTAYNHNLDTSREYYPNVITTRSYDDRLETLSHVRDAGINVCSGGIIGLGEAEEDRIGLLHTLATLPSHPESVPINALLAVKGTPLEDQKPVEIWEMIRMIGTARIVMPKAMVRLSAGRVRFSMSEQALCFLAGANSIFTGEKLLTTPNNDFDADQLMFKTLGLIPKPPSFSEDDSESENCEKVASASH